(19) United States
(12) Patent Application Publication
(10) Pub. No.: US 2017/0262580 A1
(43) Pub. Date: Sep. 14, 2017

(54) METHODS AND SYSTEMS FOR ASSESSING INFERTILITY AND OVULATORY FUNCTION DISORDERS

(71) Applicant: Celmatix Inc., New York, NY (US)

(72) Inventors: Piraye Yurttas Beim, New York, NY (US); David Emlyn Parfitt, New York, NY (US); Tina Hu-Seliger, New York, NY (US); Anthony Santistevan, New York, NY (US)

(21) Appl. No.: 15/454,726

(22) Filed: Mar. 9, 2017

Related U.S. Application Data
(60) Provisional application No. 62/306,027, filed on Mar. 9, 2016.

(51) Int. Cl. G06F 19/22; C12Q 1/68

(52) U.S. Cl. CPC G06F 19/22; C12Q 1/6883; C12Q 2600/156

(57) ABSTRACT

The present invention relates to methods and systems for assessing risk of infertility and ovarian dysfunction and/or diminished ovarian reserve and/or for determining an appropriate course of treatment. In some embodiments, the invention provides methods for assessing likelihood of ovarian dysfunction, including identifying a plurality of genetic variants that are filtered into functional biological pathways. The frequency distribution of the variants in each functional pathway is then compared to frequency distributions obtained from reference sets corresponding to each pathway. Further embodiments of the invention comprise clustering subjects based on patterns in their genetic variants, and identifying phenotypic differences with respect to ovarian dysfunction between clusters of patients.

### CROSS-REFERENCE TO RELATED APPLICATIONS

This application claims priority to and the benefit of U.S. Provisional Application No. 62/306,027, filed Mar. 9, 2016, the content of which is incorporated herein in its entirety.

### BACKGROUND

According to the Centers for Disease Control and Prevention, 6.7 million women (around 10.9%) in the United States between the ages of 15 and 44 suffer from impaired fecundity. See Chandra A, Copen C E, Stephen E H. Infertility and impaired fecundity in the United States, 1982-2010: Data from the National Survey of Family Growth. National health statistics reports; no 67. Hyattsville, Md.: National Center for Health Statistics, 2013. A woman's egg quality and number naturally begin to decline at around age 35. Reduced fecundity as a result of declining ovarian reserve and function leading up to menopause is a normal part of aging in females. However, in some women, ovarian aging happens prematurely, sometimes resulting in ovarian function disorders such as diminished ovarian reserve (DOR) or primary ovarian insufficiency (POI), which have a negative impact on fecundity. Other ovarian function disorders, such as polycystic ovary syndrome (PCOS), can also have a negative effect on fertility in women. The American Society for Reproductive Medicine (ASRM) estimates that 5-10% of all women suffer from PCOS, while 1% suffer from POI. Estimates of the prevalence of DOR vary, as DOR can be age-related, but the Center for Human Reproduction estimates that ˜10% of women have ovarian reserves that are lower than normal for their age. The Center for Disease Control (CDC) estimates that overall, 40-50% of women seeking fertility treatments have abnormal ovarian reserve measurements.

Ovarian function disorders are classified into distinct diagnoses based on the clinical manifestation of each condition. For example, DOR is a condition in which a woman's ovaries contain fewer eggs than would be expected based upon age. This can make conception more difficult and decreases the chance of conceiving with in vitro fertilization (IVF) and other fertility treatments. DOR can also result in a higher chance of miscarriage.

Another example of ovarian dysfunction is POI, a condition in which a female loses normal function of her ovaries before the age of 40. This loss of function leads to the failure of the ovaries to produce normal amounts of the hormone estrogen. Also stemming from the loss in function is the failure of the ovaries to release eggs on a regular basis. Infertility often results from POI. At the current time, there is no known treatment to restore fertility in females with this condition. One option for pursuing pregnancy for women suffering from infertility caused by POI is IVF using donor eggs or eggs that have been harvested and frozen prior to developing POI and becoming infertile.

PCOS is the most common endocrine disorder among women between the ages of 18 and 44 and manifests itself in a set of symptoms often caused by hormonal and metabolic imbalances in women, for example leading to elevated androgen levels and insulin resistance. Such changes can cause an arrest in ovarian follicular development, such that the dominant follicle fails to emerge. Eggs thus become trapped in the ovaries at varying stages of development, and cysts begin to form. PCOS can lead to a decrease in fertility, depending on the severity. Currently, there is no cure for PCOS, but various treatment options are currently in use ranging from lifestyle changes, such as weight loss and exercise, to pharmaceutical drugs, to aspiration of eggs from more mature follicles (to use during e.g., IVF). Although there are no pharmaceutical drugs currently indicated for the specific treatment of PCOS and/or improvement in fertility in PCOS patients, various drugs, such as birth control pills, metformin, and clomiphene, are currently prescribed to patients. The administration of these drugs is designed to override a patient's self-regulatory loop and force the follicles to more fully develop such that ovulation occurs.

Different ovarian function disorders frequently impact ovarian reserve and, interestingly, patients suffering from them often present a number of similar clinical symptoms (e.g., irregular menstrual cycles). These disorders could therefore be thought of as a spectrum of conditions, with particular diagnoses falling at different points along this spectrum. For example, POI represents the most extreme example of reduced ovarian reserve, thus could be placed on one end of the spectrum. Diagnoses such as DOR, and ‘ovulatory dysfunction’ could be placed further along the spectrum, with PCOS, associated with metabolic disorders and excess androgens, on the opposite end of the spectrum to POI.

Although genetic studies have shed light on molecular defects associated with ovarian function disorders, the extent of their etiologies was largely unexplored. By considering these conditions along a continuous spectrum of symptoms and clinical phenotypes, we could gain a better understanding of their etiologies, and thus better determine their impact on fertility and instruct potential treatment options. Accordingly, there is a need for improved assessment of risk of ovarian dysfunction in patients.

### SUMMARY

The invention relates to methods and systems for assessing risk of infertility and ovarian dysfunction and/or diminished ovarian reserve and/or for determining an appropriate course of treatment. In some embodiments, the invention provides methods for assessing likelihood of ovarian dysfunction, including identifying a plurality of genetic variants that are filtered into functional biological pathways. The frequency distribution of the variants in each functional pathway is then compared to frequency distributions obtained from reference sets corresponding to each pathway. Further embodiments of the invention comprise clustering subjects based on patterns in their genetic variants, and identifying phenotypic differences with respect to ovarian dysfunction between clusters of patients.

Other aspects of the invention involve methods for determining likely treatment outcomes in patients suffering from ovarian dysfunction. The invention also relates to methods for determining key genetic pathways underlying ovarian function disorders. Methods of the invention typically are implemented on a computer system having resident memory for storing data as indicated below and executable code for performing the methods taught herein.

### BRIEF DESCRIPTION OF DRAWINGS

FIG. 1 illustrates a pathway-enrichment matrix showing biological pathways with higher frequency of deleterious variants in a certain group compared to the estimated frequencies in all groups (p<0.0125). Pathways with frequencies of deleterious variants consistent with the estimated average from all groups are indicated in black (p<0.0125).

FIG. 2 illustrates gene networks within the steroid biosynthesis pathway. The largest nodes represent gene variants with significantly higher frequencies in the DOR group compared to the PCOS group.

FIG. 3 illustrates gene networks within the oogenesis pathway. The largest nodes represent gene variants with significantly higher frequencies in the PCOS group compared to the DOR group.

FIG. 4 illustrates gene networks within the ovarian follicular development pathway. The largest nodes represent gene variants with higher frequencies in both DOR and PCOS groups compared to the control group.

FIG. 5 illustrates common and unique genetic pathways contributing to ovarian function disorders.

FIG. 6 represents a diagram of a system of the invention.

FIG. 7 illustrates how patients form distinct clusters with respect to their distribution of fertility-related single nucleotide variants.

### DETAILED DESCRIPTION

The invention relates to methods and systems for assessing likelihood of abnormal ovarian function and reserve, and infertility in a female subject and informing course of treatment thereof. Aspects of the invention include identifying genetic biomarkers and genetic pathways underlying ovarian dysfunction. These biomarkers and pathways can be utilized to provide accurate risk profiles that can inform downstream diagnostic tests and treatments that may benefit the individual.

In one embodiment, the methods of the invention involve obtaining nucleic acids from a sample, the sample being obtained from a female subject. Nucleic acids from the sample are then sequenced to generate sequence reads. The sequence reads can then be compared to a reference (e.g., hg18) to identify single nucleotide polymorphisms (SNPs) or single nucleotide variants (SNVs), copy number variants, structural variants, and other clinically-relevant variants. The detected variants are further analyzed to identify which ones are or might be deleterious and, specifically, which ones might be fertility-centric.

In one aspect, the methods of the invention comprise obtaining a sample, e.g. a tissue or body fluid, which is suspected to include a biomarker indicating the likelihood of abnormal ovarian reserve and/or function. The sample may be collected in any clinically-acceptable manner. A tissue is a mass of connected cells and/or extracellular matrix material, e.g. skin tissue, endometrial tissue, nasal passage tissue, CNS tissue, neural tissue, eye tissue, liver tissue, kidney tissue, placental tissue, mammary gland tissue, placental tissue, gastrointestinal tissue, musculoskeletal tissue, genitourinary tissue, bone marrow, and the like, derived from, for example, a human or other mammal and includes the connecting material and the liquid material in association with the cells and/or tissues. A body fluid is a liquid material derived from, for example, a human or other mammal. Such body fluids include, but are not limited to, mucous, blood, plasma, serum, serum derivatives, bile, blood, maternal blood, phlegm, saliva, sweat, amniotic fluid, menstrual fluid, mammary fluid, follicular fluid of the ovary, fallopian tube fluid, peritoneal fluid, urine, and cerebrospinal fluid (CSF), such as lumbar or ventricular CSF. A sample may also be a fine needle aspirate or biopsied tissue. A sample also may be media containing cells or biological material. In certain embodiments, infertility-associated genes or gene products may be found in reproductive cells or tissues, such as gametic cells, gonadal tissue, fertilized embryos, and placenta. In certain embodiments, the sample is drawn whole blood.

Nucleic acids are extracted from the sample according to methods known in the art. See for example, Maniatis, et al., Molecular Cloning: A Laboratory Manual, Cold Spring Harbor, N.Y., pp. 280-281, 1982, the contents of which are incorporated by reference herein in their entirety. In certain embodiments, a genomic sample is collected from a subject followed by enrichment for genetic regions or genetic fragments of interest, for example before hybridization to a nucleotide array designed to assay ovarian reserve genes or gene fragments of interest. The sample may be enriched for variants in genes or vary by expression levels of genes of interest using methods known in the art, such as hybrid capture. See for examples, Lapidus (U.S. Pat. No. 7,666,593), the content of which is incorporated by reference herein in its entirety.

Genetic data can be obtained, for example, by conducting an assay that detects a variant in an infertility-associated genetic region or abnormal expression of an infertility-associated genetic region. The presence of certain variants in those genetic regions or abnormal expression levels of those genetic regions is indicative of fertility- or fecundity-related disorders. Exemplary variants include, but are not limited to, a single nucleotide polymorphism, a single nucleotide variant, a deletion, an insertion, an inversion, a genetic rearrangement, a copy number variation, chromosomal microdeletion, genetic mosaicism, karyotype abnormality, or a combination thereof.

In particular embodiments, the assay is conducted on genetic regions of fertility related genes, or more specifically, genetic regions related to ovarian reserve and/or function. Detailed descriptions of conventional methods, such as those employed to make and use nucleic acid arrays, amplification primers, hybridization probes, and the like are found in standard laboratory manuals such as: Genome Analysis: A Laboratory Manual Series (Vols. I-IV), Cold Spring Harbor Laboratory Press; PCR Primer: A Laboratory Manual, Cold Spring Harbor Laboratory Press; and Sambrook, J et al., (2001) Molecular Cloning: A Laboratory Manual, 2nd ed. (Vols. 1-3), Cold Spring Harbor Laboratory Press. Custom nucleic acid arrays are commercially available from, e.g., Affymetrix (Santa Clara, Calif.), Applied Biosystems (Foster City, Calif.), and Agilent Technologies (Santa Clara, Calif.).

Methods of detecting genomic variants are known in the art. In certain embodiments, a known single nucleotide polymorphism at a particular position can be detected by single base extension for a primer that binds to the sample DNA adjacent to that position. See for example Shuber et al. (U.S. Pat. No. 6,566,101), the content of which is incorporated by reference herein in its entirety. In other embodiments, a hybridization probe might be employed that overlaps the SNP of interest and selectively hybridizes to sample nucleic acids containing a particular nucleotide at that position. See for example Shuber et al. (U.S. Pat. Nos. 6,214,558 and 6,300,077), the content of which is incorporated by reference herein in its entirety.

In particular embodiments, nucleic acids are sequenced in order to detect variants (i.e., mutations) in the nucleic acids compared to wild-type and/or non-mutated forms of the sequence. Methods of detecting sequence variants are known in the art, and sequence variants can be detected by any sequencing method known in the art e.g., ensemble sequencing or single molecule sequencing.

Sequencing may be by any method known in the art. DNA sequencing techniques include classic dideoxy sequencing reactions (Sanger method) using labeled terminators or primers and gel separation in slab or capillary, sequencing by synthesis using reversibly terminated labeled nucleotides, pyrosequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Sequencing of separated molecules has more recently been demonstrated by sequential or single extension reactions using polymerases or ligases as well as by single or sequential differential hybridizations with libraries of probes.

One conventional method to perform sequencing is by chain termination and gel separation, as described by Sanger et al., Proc Natl. Acad. Sci. USA, 74(12): 5463 67 (1977). Another conventional sequencing method involves chemical degradation of nucleic acid fragments. See, Maxam et al., Proc. Natl. Acad. Sci., 74: 560 564 (1977). Finally, methods have been developed based upon sequencing by hybridization. See, e.g., Harris et al., (U.S. patent application number 2009/0156412). The content of each reference is incorporated by reference herein in its entirety.

A sequencing technique that can be used in the methods of the provided invention includes, for example, Helicos True Single Molecule Sequencing (tSMS) (Harris T. D. et al. (2008) Science 320:106-109), incorporated herein by reference; see also, e.g., Lapidus et al. (U.S. Pat. No. 7,169,560), Lapidus et al. (U.S. patent application number 2009/0191565), Quake et al. (U.S. Pat. No. 6,818,395), Harris (U.S. Pat. No. 7,282,337), Quake et al. (U.S. patent application number 2002/0164629), and Braslaysky, et al., PNAS (USA), 100: 3960-3964 (2003), the contents of each of these references is incorporated by reference herein in its entirety. Another example of a DNA sequencing technique that can be used in the methods of the provided invention is 454 sequencing (Roche) (Margulies, M et al. 2005, Nature, 437, 376- 380).

Another example of a DNA sequencing technique that can be used in the methods of the provided invention is SOLiD technology (Applied Biosystems). Another example of a DNA sequencing technique that can be used in the methods of the provided invention is Ion Torrent sequencing (U.S. patent application numbers 2009/0026082, 2009/0127589, 2010/0035252, 2010/0137143, 2010/0188073, 2010/0197507, 2010/0282617, 2010/0300559), 2010/0300895, 2010/0301398, and 2010/0304982), the content of each of which is incorporated by reference herein in its entirety.

Another example of a sequencing technology that can be used in the methods of the provided invention is next-gen sequencing, such as Illumina sequencing, using Illumina HiSeq sequencers. Illumina sequencing is based on the amplification of DNA on a solid surface using fold-back PCR and anchored primers. Genomic DNA is fragmented, and adapters are added to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated.

Another example of a sequencing technology that can be used in the methods of the provided invention includes the single molecule, real-time (SMRT) technology of Pacific Biosciences. In SMRT, each of the four DNA bases is attached to one of four different fluorescent dyes. These dyes are phospholinked. A single DNA polymerase is immobilized with a single molecule of template single stranded DNA at the bottom of a zero-mode waveguide (ZMW). A ZMW is a confinement structure that enables observation of incorporation of a single nucleotide by DNA polymerase against the background of fluorescent nucleotides that rapidly diffuse in and out of the ZMW (in microseconds). It takes several milliseconds to incorporate a nucleotide into a growing strand. During this time, the fluorescent label is excited and produces a fluorescent signal, and the fluorescent tag is cleaved off. Detection of the corresponding fluorescence of the dye indicates which base was incorporated. The process is repeated.

Another example of a sequencing technique that can be used in the methods of the provided invention is nanopore sequencing (Soni G V and Meller A. (2007) Clin Chem 53: 1996-2001, incorporated herein by reference). Another example of a sequencing technique that can be used in the methods of the provided invention involves using a chemical-sensitive field effect transistor (chemFET) array to sequence DNA (for example, as described in US Patent Application Publication No. 20090026082 and incorporated by reference). Another example of a sequencing technique that can be used in the methods of the provided invention involves using an electron microscope (Moudrianakis E. N. and Beer M. Proc Natl Acad Sci USA. 1965 March; 53:564-71, incorporated herein by reference).

If the nucleic acid from the sample is degraded or only a minimal amount of nucleic acid can be obtained from the sample, PCR can be performed on the nucleic acid in order to obtain a sufficient amount of nucleic acid for sequencing (See e.g., Mullis et al. U.S. Pat. No. 4,683,195, the contents of which are incorporated by reference herein in its entirety).

Sequencing by any of the methods described above and known in the art produces sequence reads. Sequence reads can be analyzed to call variants by any number of methods known in the art. Variant calling can include aligning sequence reads to a reference (e.g. hg18) and reporting single nucleotide (SNP) alleles. An example of methods for analyzing sequence reads and calling variants includes standard Genome Analysis Toolkit (GATK) methods. See The Genome Analysis Toolkit: a MapReduce framework for analyzing next-generation DNA sequencing data, Genome Res 20(9):1297-1303, the contents of each of which are incorporated by reference. GATK is a software package for analysis of high-throughput sequencing data capable of identifying variants, including SNPs.

SNP alleles can be reported in a format such as a Sequence Alignment Map (SAM) or a Variant Call Format (VCF) file. Some background may be found in Li & Durbin, 2009, Fast and accurate short read alignment with Burrows-Wheeler Transform. Bioinformatics 25:1754-60 and McKenna et al., 2010. Variant calling produces results (“variant calls”) that may be stored as a sequence alignment map (SAM) or binary alignment map (BAM) file—comprising an alignment string (the SAM format is described, e.g., in Li, et al., The Sequence Alignment/Map format and SAMtools, Bioinformatics, 2009, 25(16):2078-9). Additionally or alternatively, output from the variant calling may be provided in a variant call format(VCF) file, e.g., in report. A typical VCF file will include a header section and a data section. The header contains an arbitrary number of meta-information lines, each starting with characters ‘##’, and a TAB delimited field definition line starting with a single ‘#’ character. The field definition line names eight mandatory columns, and the body section contains lines of data populating the columns defined by the field definition line. The VCF format is described in Danecek et al., 2011, The VCF and VCFtools, Bioinformatics 27(15):2156-2158. Further discussion may be found in U.S. Pub. 2013/0073214; U.S. Pub. 2013/0345066; U.S. Pub. 2013/0311106; U.S. Pub. 2013/0059740; U.S. Pub. 2012/0157322; U.S. Pub. 2015/0057946 and U.S. Pub. 2015/0056613, each incorporated by reference.

Once the SNPs have been identified, deleterious SNPs can be determined by any number of methods known in the art. One example of a method for determining deleterious SNPs is through the use of SnpEff, a genetic variant annotation and effect prediction toolbox. SnpEff is capable of rapidly categorizing the effects of SNPs and other variants in whole genome sequences. See, Cingolani et al., A program for annotating and predicting the effects of single nucleotide polymorphisms, SnpEff: SNPs in the genome of Drosophila melanogaster strain w^{1118;} iso-2; iso-3; Landes Bioscience, 6:2, 1-13; April/May/June 2012, incorporated herein by reference.

Upon identification of deleterious variants, the variants can be filtered for those that are fertility-centric. One of ordinary skill in the art would understand that both molecular and computational approaches are available for filtering variants. One of ordinary skill in the art would also understand how to filter deleterious variants for fertility centric genes (e.g. by comparing to a known database, through the use of ANOVA technology, through the use of multivariant analysis). It is to be understood that various fertility-centric bioinformatics pipelines incorporating pathway analysis tools can be used to filter deleterious variants in accordance with the invention.

In one aspect of the invention, genes of interest can be annotated into functional biological pathways using any method known in the art. One example of a pathway analysis tool for gene annotation includes the Database for Annotation, Visualization and Integrated Discover (DAVID). Nature Protocols 2009; 4(1):44; and Nucleic Acids Res. 2009; 37(1):1, incorporated herein by reference. The correlation between variants in functional biological pathways and fertility-related phenotypes can be analyzed using any known statistical methods. In one embodiment, frequency distribution of deleterious variants in each pathway can be determined using a paired-Wilcoxon test. Various implementations of the Wilcoxon test include ALGLIB in C++, C#, Delphi, Visual Basic, etc. (http://www.alglib.net/aboutus.php); the R Project for Statistical Computing (https://www.r-project.org/); GNU Octave (https://www.gnu.org/software/octave/); and SciPy in Python.

In other embodiments of the invention, the correlation between variants in functional biological pathways and fertility-related phenotypes can be assessed through sequence kernel association testing (SKAT). See Wu MC, Lee S, Cai T, Li Y, Boehnke M, Lin X. Rare-Variant Association Testing for Sequencing Data with the Sequence Kernel Association Test. American Journal of Human Genetics. 2011;89(1):82-93. doi:10.1016/j.ajhg.2011.05.029, incorporated herein by reference.

SKAT is a variant—or gene-set level methodology for testing whether variant-sets are associated with phenotypes (continuous or discrete) of interest. Variant-sets can be defined by genes, functional biological pathways, or genomic regions, etc. These sets are required to be defined prior to performing a SKAT analysis. Gene sets can be defined in any number of ways, such as through use of a fertility-centric database, as described in more detail below. Moreover, through utilizing different kernels, SKAT may test for an association between two or more variants within a variant-set and a phenotype of interest.

The SKAT method lends an improvement over single SNV-level analyses by reducing the burden of correcting for multiple comparisons, thereby increasing the power to detect true associations. SKAT aggregates variant-level score test statistics, or score statistics based on the interaction between two or more variants, within a variant-set to compute a p-value for variant-set level significance. Additionally, SKAT allows for the incorporation of covariates, which allows the method to identify if SNV-sets are correlated with phenotypes of interest even after adjusting for other variables.

SKAT makes no assumption as to the direction of the effect of individual variants, or groups of variants, on the phenotype, and as such, is a powerful approach for detecting variant-set level associations in cases where individual variants, or groups of variants, within a category may have differential effects on the phenotype of interest. SKAT assumes that the effects of variants, or groups of variants, on the phenotype follow a distribution with a mean of zero (i.e., no effect on the phenotype) and variance σ². SKAT utilizes a variance-components test of the hypothesis that the variance of the variant, or groups of variants, effect is non-zero; i.e., σ²≠0, which provides evidence that there is a variant-set level association.

Because SKAT only provides a p-value for the evidence of an association between the variant-set and the phenotype of interest, but no measure of the magnitude or direction of this effect, burden testing can be completed to enhance the results of the SKAT analysis.

Burden tests collapse individual variant-level genetic information, or groups of variants, to the variant-set level (e.g., gene—or functional pathway—level). For example, each patient can be assigned a genetic burden score within a given functional pathway by computing a sum score of the total number of deleterious variants each patient had within each pathway. Additionally, patients may be assigned a genetic burden score based on the number of times variants co-occur within a pathway. Burden scores can then be incorporated into standard regression models, which can also control for clinical metrics known to be associated with the phenotype of interest.

Accordingly, by adjusting models according to SKAT-analysis results, one is able to see whether there is statistical evidence that genomic information, at the category level (e.g. functional biological pathway level), provides additional information beyond known clinical metrics that is sufficient to significantly affect the model, and therefore be associated with the phenotype of interest.

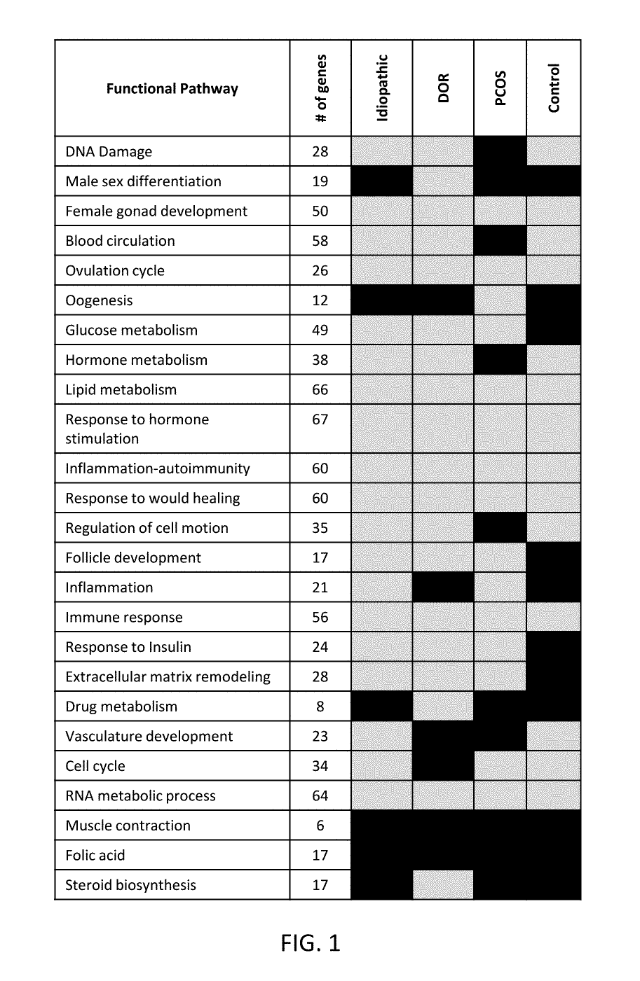

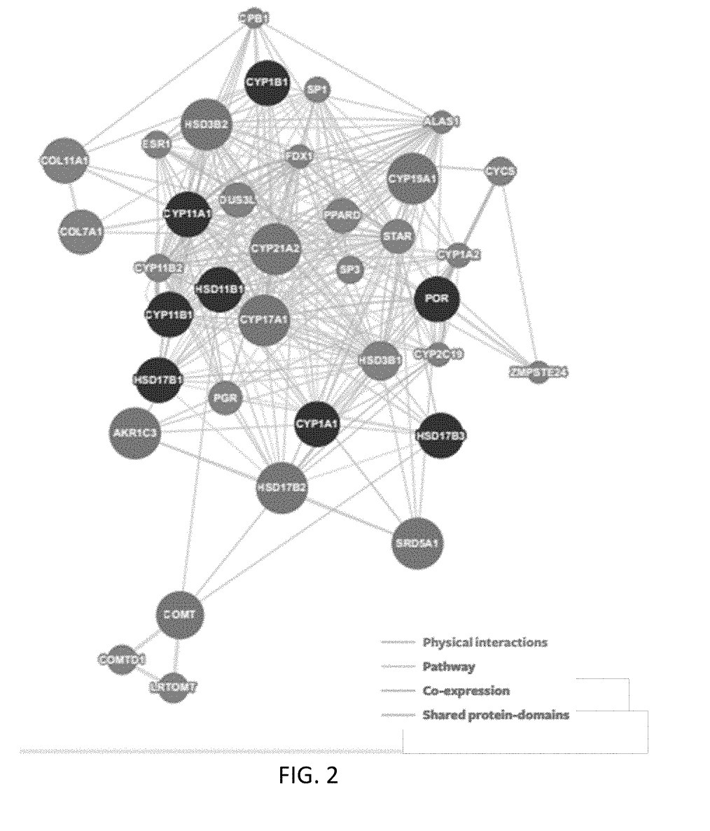

In one embodiment, genetic variants, such as SNPs, identified using methods of the invention can be used as biomarkers to assess the likelihood of ovarian dysfunction, and thus infertility, and can also be used to guide course of treatment. Biomarkers according to the invention include variations in any number of fertility-centric genes. Fertility-centric genes can be any gene that affects the reserve fertility in females and/or males. The genes may also fall within any one of the pathways shown in FIG. 1. Exemplary genes include, but are not limited to, the genes shown in FIGS. 2-4 and listed in Table 4 below.

In one embodiment, methods of targeting treatment upon assessment of ovarian function in a female subject are provided. For instance, with respect to POI, although most patients with POI experience complete infertility, early diagnosis of the disorder can indicate that the patient may be able to achieve pregnancy and live birth by resorting to fertility treatments, including egg cryo-preservation, ovarian cortex cryo-preservation, and/or IVF before their conditions worsens. In other situations, a diagnosis of POI may indicate that pregnancy and live birth cannot be achieved using the female's own eggs, but can be achieved by IVF procedures using a donor egg(s). With respect to DOR, the patient may be able to achieve pregnancy and live birth using their own eggs by various treatment options such as, for example and not limited to, supplementation with the androgen dehydroepiandrosterone (DHEA), IVF, other fertility treatments known in the art, and combinations thereof. In the case of risk of DOR and/or POI, immune function modulating therapies such as treatment with TNF-inhibitors. Also in the case of risk of DOR and/or POI, therapies targeting inflammation include surgical and pharmacological interventions. In the case of PCOS, for example, the patient can be prescribed various medications that can assist with the development of follicles, and thus trigger ovulation.

In one aspect, assessment and analysis of ovarian function and/or fertility can include the incorporation of fertility-associated phenotypic and/or environmental characteristics. Exemplary traits are provided in Table 1 below.

TABLE 1
Phenotypic and environmental variables impacting fertility success

Cholesterol levels on different days of the menstrual cycle
Age of first menses for patient and female blood relatives (e.g. sisters, mother,
grandmothers)
Age of menopause for female blood relatives (e.g. sisters, mother, grandmothers)
Number of previous pregnancies (biochemical/ectopic/clinical/fetal heart beat detected, live
birth outcomes), age at the time, and outcome for patient and female blood relatives (e.g.
sisters, mother, grandmothers)
Diagnosis of PCOS
History of hydrosalpinx or tubal occlusion
History of endometriosis, pelvic pain, or painful periods
Cancer history/type of cancer/treatment/outcome for patient and female blood relatives (e.g.
sisters, mother, grandmothers)
Age that sexual activity began, current level of sexual activity
Smoking history for patient and blood relatives
Travel schedule/number of flying hours a year/time difference changes of more than 3 hours
(Jetlag and Flight-associated Radiation Exposure)
Nature of periods (length of menses, length of cycle)
Biological age (number of years since first menses)
Birth control use
Drug use (illegal or legal)
Body mass index (current, lowest ever, highest ever)
History of polyps
History of hormonal imbalance
History of amenorrhoea
History of eating disorders
Alcohol consumption by patient or blood relatives
Details of mother's pregnancy with patient (i.e. measures of uterine environment): any drugs
taken, smoking, alcohol, stress levels, exposure to plastics (i.e. Tupperware), composition of
diet (see below)
Sleep patterns: number of hours a night, continuous/overall
Diet: meat, organic produce, vegetables, vitamin or other supplement consumption, dairy
(full fat or reduced fat), coffee/tea consumption, folic acid, sugar (complex, artificial,
simple), processed food versus home cooked.
Exposure to plastics: microwave in plastic, cook with plastic, store food in plastic, plastic
water or coffee mugs.
Water consumption: amount per day, format: straight from the tap, bottled water (plastic or
bottle), filtered (type: e.g. Britta/Pur)
Residence history starting with mother's pregnancy: location/duration
Environmental exposure to potential toxins for different regions (extracted from government
monitoring databases)
Health metrics: autoimmune disease, chronic illness/condition
Pelvic surgery history
Life time number of pelvic X-rays
History of sexually transmitted infections: type/treatment/outcome
Reproductive hormone levels: follicle stimulating hormone, anti-Müllerian hormone,
estrogen, progesterone
Stress
Thickness and type of endometrium throughout the menstrual cycle.
Age
Height
Fertility treatment history and details: history of hormone stimulation, brand of drugs used,
basal antral follicle count, follicle count after stimulation with different protocols,
number/quality/stage of retrieved oocytes/development profile of embryos resulting from in
vitro insemination (natural or ICSI), details of IVF procedure (which clinic,
doctor/embryologist at clinic, assisted hatching, fresh or thawed oocytes/embryos, embryo
transfer (blood on the catheter/squirt detection and direction on ultrasound), number of
successful and unsuccessful IVF attempts
Morning sickness during pregnancy
Breast size before/during/after pregnancy
History of ovarian cysts
Twin or sibling from multiple birth (mono-zygotic or di-zygotic)
Male factor infertility for reproductive partner: Semen analysis (count, motility,morphology),
Vasectomy, male cancer, smoking, alcohol, diet, STIs
Blood type
DES exposure in utero
Past and current exercise/athletic history
Levels of phthalates, including metabolites:
MEP-monoethyl phthalate, MECPP-mono(2-ethyl-5-carboxypentyl) phthalate, MEHHP-
mono(2-ethyl-5-hydroxyhexyl) phthalate, MEOHP-mono(2-ethyl-5-ox-ohexyl) phthalate,
MBP-monobutyl phthalate, MBzP-monobenzyl phthalate, MEHP-mono(2-ethylhexyl)
phthalate, MiBP-mono-isobutyl phthalate, MCPP-mono(3-carboxypropyl) phthalate,
MCOP-monocarboxyisooctyl phthalate, MCNP-monocarboxyisononyl phthalate
Familial history of Premature Ovarian Failure/Insufficiency
Autoimmunity history-Antiadrenal antibodies (anti-21-hydroxylase antibodies), antiovarian
antibodies, antithyroid anitibodies (anti-thyroid peroxidase, antithyroglobulin)
Hormone levels: Leutenizing hormone (using immunofluorometric assay),
Δ4-Androstenedione (using radioimmunoassay), Dehydroepiandrosterone (using
radioimmunoassay), and Inhibin B (commercial ELISA)
Number of years trying to conceive
Dioxin and PVC exposure
Hair color
Nevi (moles)
Lead, cadmium, and other heavy metal exposure
For a particular ART cycle: the percentage of oocytes that were abnormally fertilized, if
assisted hatching was performed, if anesthesia was used, average number of cells contained
by the embryo at the time of cryopreservation, average degree of expansion for blastocyst
represented as a score, average degree of expansion of a previously frozen embryo
represented as a score, embryo quality metrics including but not limited to degree of cell
fragmentation and visualization of a or organization/number of cells contained in the inner
cell mass, the fraction of overall embryos that make it to the blastocyst stage of development,
the number of embryos that make it to the blastocyst stage of development, use of birth
control, the brand name of the hormones used in ovulation induction, hyperstimulation
syndrome, reason for cancelation of a treatment cycle, chemical pregnancy detected, clinical
pregnancy detected, count of germinal vesicle containing oocytes upon retrieval, count of
metaphase I stage oocytes upon retrieval, count of metaphase II stage oocytes upon retrieval,
count of embryos or oocytes arrested in development and the stage of development or day of
development post oocyte retrieval, number of embryos transferred and date in days post-
oocyte retrieval that the embryos were transferred, how many embryos were cryopreserved
and at what stage of development

Information regarding the fertility-associated phenotypic traits, such as those listed in Table 1, can be obtained by any means known in the art. In many cases, such information can be obtained from a questionnaire completed by the subject that contains questions regarding certain fertility-associated phenotypic traits. Additional information can be obtained from a questionnaire completed by the subject's partner and blood relatives. The questionnaire includes questions regarding the subject's fertility-associated phenotypic traits, such as his or her age, smoking habits, or frequency of alcohol consumption. Information can also be obtained from the medical history of the subject, as well as the medical history of blood relatives and other family members. Additional information can be obtained from the medical history and family medical history of the subject's partner. Medical history information can be obtained through analysis of electronic medical records, paper medical records, a series of questions about medical history included in the questionnaire, and a combination thereof.

In other embodiments, an assay specific to a phenotypic trait or an environmental exposure of interest is used. Such assays are known to those of skill in the art, and may be used with methods of the invention. For example, the hormones used in birth control pills (estrogen and progesterone) may be detected from a urine or blood test. Venners et al. (Hum. Reprod. 21(9): 2272-2280, 2006) reports assays for detecting estrogen and progesterone in urine and blood samples. Venner also reports assays for detecting the chemicals used in fertility treatments.

Similarly, illicit drug use may be detected from a tissue or body fluid, such as hair, urine, sweat, or blood, and there are numerous commercially available assays (LabCorp) for conducting such tests. Standard drug tests look for ten different classes of drugs, and the test is commercially known as a “10-panel urine screen”. The 10-panel urine screen consists of the following: 1. Amphetamines (including Methamphetamine) 2. Barbiturates 3. Benzodiazepines 4. Cannabinoids (THC) 5. Cocaine 6. Methadone 7. Methaqualone 8. Opiates (Codeine, Morphine, Heroin, Oxycodone, Vicodin, etc.) 9. Phencyclidine (PCP) 10. Propoxyphene. Use of alcohol can also be detected by such tests.

Numerous assays can be used to test a patient's exposure to plastics (e.g., Bisphenol A (BPA)). BPA is most commonly found as a component of polycarbonates (about 74% of total BPA produced) and in the production of epoxy resins (about 20%). As well as being found in a myriad of products including plastic food and beverage contains (including baby and water bottles), BPA is also commonly found in various household appliances, electronics, sports safety equipment, adhesives, cash register receipts, medical devices, eyeglass lenses, water supply pipes, and many other products. Assays for testing blood, sweat, or urine for presence of BPA are described, for example, in Genuis et al. (Journal of Environmental and Public Health, Volume 2012, Article ID 185731, 10 pages, 2012).

Various known association analysis and statistical pattern recognition methods can be used in conjunction with the present invention to incorporate genetic, phenotypic and/or environmental characteristics to assess the likelihood of ovarian dysfunction and/or infertility in subjects. Suitable methods include, without limitation, logistic regression, ordinal logistic regression, linear or quadratic discriminant analysis, clustering, principal component analysis, multiple correspondence analysis, nearest neighbor classifier analysis, random forests, artificial neural networks, and Cox proportional hazards regression.

In one embodiment of the invention, multiple correspondence analysis (MCA) is utilized to reveal patterns in the distribution of SNVs in a patient or group of patients. Multiple correspondence analysis is a subset of techniques used to reveal patterning in complex datasets. As a non-limiting example, a set of 50 SNVs may be subjected to MCA, which may uncover 4 common dimensions along which these 50 SNVs can be described. Those dimensions may correspond to genes, pathways, or any other biologically meaningful parameter that may be linked with the variants. Rather than analyzing the association between each SNV and phenotypes of interest, the values along each of the 4 dimensions are correlated with phenotypes of interest, thus lowering the dimension of the problem from 50 to 4 and identifying meaningful sets of SNVs.

Patients may then be clustered based on their values in the dimensions discovered by MCA, or other dimensionality reduction techniques, in order to uncover sets of SNVs which correlate with phenotypes of interest including, but not limited to, DOR, POI or PCOS. In one method of the invention, hierarchical clustering may be used to cluster patients based on the dimensions uncovered by MCA. Hierarchical clustering based on dimensions discovered by MCA algorithmically identifies clusters of patients that have similar values in the dimensions discovered by MCA, and therefore have similar SNV sets.

In addition, haplotypic relationships among groups of genetic variants can be estimated using programs such as Haploscore. Alternatively, programs such as Haploview and Phase can be used to estimate haplotype frequencies and then further analysis such as Chi square test can be performed. Logistic regression analysis may be used to generate an odds ratio and relative risk for each characteristic.

Methods of logistic regression are described, for example in, Ruczinski (Journal of Computational and Graphical Statistics 12:475-512, 2003); Agresti (An Introduction to Categorical Data Analysis, John Wiley & Sons, Inc., 1996, New York, Chapter 8); and Yeatman et al. (U.S. patent application number 2006/0195269), the content of each of which is hereby incorporated by reference in its entirety.

Other algorithms for analyzing associations are known. For example, the stochastic gradient boosting is used to generate multiple additive regression tree (MART) models to predict a range of outcome probabilities. Each tree is a recursive graph of decisions the possible consequences of which partition patient parameters; each node represents a question (e.g., is the FSH level greater than x?) and the branch taken from that node represents the decision made (e.g. yes or no). The choice of question corresponding to each node is automated. A MART model is the weighted sum of iteratively produced regression trees. In each iteration, a regression tree is fitted according to a criterion in which the samples more involved in the prediction error are given priority. This tree is added to the existing trees, the prediction error is recalculated, and the cycle continues, leading to a progressive refinement of the prediction. The strengths of this method include analysis of many variables without knowledge of their complex interactions beforehand.

A different approach called the generalized linear model, expresses the outcome as a weighted sum of functions of the predictor variables. The weights are calculated based on least squares or Bayesian methods to minimize the prediction error on the training set. A predictor's weight reveals the effect of changing that predictor, while holding the others constant, on the outcome. In cases where one or more predictors are highly correlated, in a phenomenon known as collinearity, the relative values of their weights are less meaningful; steps must be taken to remove that collinearity, such as by excluding the nearly redundant variables from the model. Thus, when properly interpreted, the weights express the relative importance of the predictors. Less general formulations of the generalized linear model include linear regression, multiple regression, and multifactor logistic regression models, and are highly used in the medical community as clinical predictors.

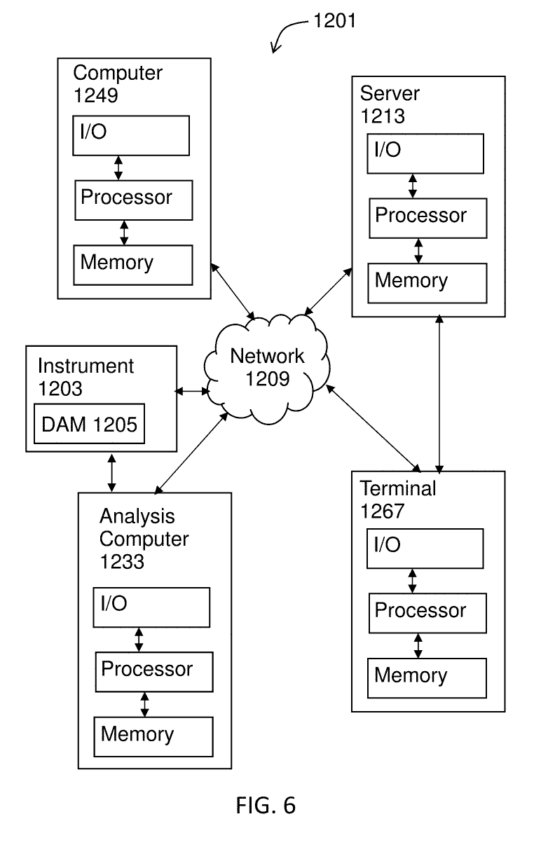

As one skilled in the art would recognize as necessary or best-suited for performance of the methods of the invention, a computer system(s) or machine(s) can be used. FIG. 6 gives a diagram of a system 1201 according to embodiments of the invention. System 1201 may include an analysis instrument 1203 which may be, for example, a sequencing instrument (e.g., a HiSeq 2500 or a MiSeq by Illumina). Instrument 1203 includes a data acquisition module 1205 to obtain results data such as sequence read data. Instrument 1203 may optionally include or be operably coupled to its own, e.g., dedicated, analysis computer 1233 (including an input/output mechanism, one or more processor, and memory). Additionally or alternatively, instrument 1203 may be operably coupled to a server 1213 or computer 1249 (e.g., laptop, desktop, or tablet) via a network 1209.

Computer 1249 includes one or more processors and memory as well as an input/output mechanism. Where methods of the invention employ a client/server architecture, steps of methods of the invention may be performed using the server 1213, which includes one or more of processors and memory, capable of obtaining data, instructions, etc., or providing results via an interface module or providing results as a file. The server 1213 may be engaged over the network 1209 by the computer 1249 or the terminal 1267, or the server 1213 may be directly connected to the terminal 1267, which can include one or more processors and memory, as well as an input/output mechanism.

In system 1201, each computer preferably includes at least one processor coupled to a memory and at least one input/output (I/O) mechanism.

A processor will generally include a chip, such as a single core or multi-core chip, to provide a central processing unit. A process may be provided by a chip from Intel or AMD.

Memory can include one or more machine-readable devices on which is stored one or more sets of instructions (e.g., software) which, when executed by the processor(s) of any one of the disclosed computers can accomplish some or all of the methodologies or functions described herein. The software may also reside, completely or at least partially, within the main memory and/or within the processor during execution thereof by the computer system. Preferably, each computer includes a non-transitory memory such as a solid state drive, flash drive, disk drive, hard drive, etc. While the machine-readable devices can in an exemplary embodiment be a single medium, the term “machine-readable device” should be taken to include a single medium or multiple media (e.g., a centralized or distributed database, and/or associated caches and servers) that store the one or more sets of instructions and/or data. These terms shall also be taken to include any medium or media that are capable of storing, encoding, or holding a set of instructions for execution by the machine and that cause the machine to perform any one or more of the methodologies of the present invention. These terms shall accordingly be taken to include, but not be limited to one or more solid-state memories (e.g., subscriber identity module (SIM) card, secure digital card (SD card), micro SD card, or solid-state drive (SSD)), optical and magnetic media, and/or any other tangible storage medium or media.

A computer of the invention will generally include one or more I/O device such as, for example, one or more of a video display unit (e.g., a liquid crystal display (LCD) or a cathode ray tube (CRT)), an alphanumeric input device (e.g., a keyboard), a cursor control device (e.g., a mouse), a disk drive unit, a signal generation device (e.g., a speaker), a touchscreen, an accelerometer, a microphone, a cellular radio frequency antenna, and a network interface device, which can be, for example, a network interface card (NIC), Wi-Fi card, or cellular modem.

Other embodiments are within the scope and spirit of the invention. For example, due to the nature of software, functions described above can be implemented using software, hardware, firmware, hardwiring, or combinations of any of these. Features implementing functions can also be physically located at various positions, including being distributed such that portions of functions are implemented at different physical locations.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore

### EXAMPLE 1

In this example, whole genome sequencing and proprietary bioinformatics pipelines were used to identify genetic pathways altered in various ovarian disorders.

### Study Design and Methodology

The study subjects consisted of 231 women seeking fertility treatment at five academic and private fertility clinics in the US. Women in the cohort were diagnosed with PCOS/ovulatory dysfunction, DOR, or received an idiopathic diagnosis. As a control, women with tubal factor, women whose partners were diagnosed with male factor, and women undergoing IVF for reasons other than fertility (e.g. same sex couples, egg donation, elective cryopreservation) were included. For each group, the number of women, average age, BMI, and basal antral follicle count (BAFC) are summarized in Tables 2 and 3.

TABLE 2
Summary statistics of analyzed patients

| | Number | | |
|---|---|---|---|
| | of patients | Average | Average |
| Diagnosis Group | (N) | Age* | BMI* |
| Idiopathic | 80 | 33 | 23.6 |
| DOR | 71 | 34 | 24.5 |
| PCOS/Ovulatory Dysfunction | 37 | 31 | 25.6 |
| Control | 43 | 33 | 24.9 |

*There were no statistically significant differences in age and BMI between groups.

TABLE 3
Basal antral follicle statistics of analyzed patients

| | Average | Average | Average |
|---|---|---|---|
| | Initial | Lowest | Highest |
| Diagnosis Group | bAFC | BAFC | BAFC |
| Idiopathic | 16 | 11 | 21 |
| DOR | 9 | 6 | 12 |
| PCOS/Ovulatory Dysfunction | 24 | 16 | 28 |
| Control | 13 | 10 | 17 |

BAFC numbers obtained from the patients provide quantitative evidence that patients in each of the groups have differences in their ovarian reserve that qualify them to be in the specific group. As can be seen, DOR patients typically have the lowest BAFC while PCOS patients typically have the highest BAFC.

DNA Sequencing Analysis: Whole blood samples were taken from each of the study subjects. Genomic DNA was extracted from the whole blood. Whole genome sequences (with an average read depth of 30X) were generated using Illumina HiSeq platform. The sequences generated were then analyzed using GATK standard methods. Single nucleotide polymorphisms (SNPs), or variants, predicted to disrupt gene function were identified using SNPeff, a variant effect prediction tool. A fertility-centric bioinformatics pipeline that incorporates pathway analysis tools was used to filter the SNPs. The Database for Annotation, Visualization and Integrated Discovery DAVID pathway analysis tool was used for gene annotation into functional biological pathways.

Pathway Enrichment Analysis: The frequency distribution of deleterious variants in each pathway was determined for each patient group and compared to their estimated frequency across all patient groups using paired-Wilcoxon test. The Median-polish method was applied to estimate the frequency of a variant across all patient groups. P-values <0.0125 (Bonferonni correction for multiple testing, 0.05/4) were considered statistically significant.

### Results

Whole genome sequences were obtained from 4 female patient groups: women with idiopathic infertility, women diagnosed with DOR, women diagnosed with PCOS/ovulatory dysfunction, and a control group.

By focusing on a curated list of 400 fertility-related genes, 5,630 deleterious gene variants were identified across all patients. By annotating the fertility-related genes and pooling gene variants into functional biological pathways instead of comparing the frequency of the individual deleterious variants across patient groups, it was found that most deleterious variants clustered into genes operating 25 functional biological pathways. FIG. 1 summarizes the identified pathways and the corresponding number of fertility genes in each pathway.

Once the pathways were identified, the enrichment of deleterious variants within them was compared between the four patient groups. It was found that the pathways more likely to be altered in DOR patients versus PCOS patients include male sex differentiation, steroid hormone biosynthesis, and drug metabolism. The pathways carrying more deleterious variants in PCOS patients compared to DOR patients were inflammation and oogenesis. Ovarian follicular development, glucose metabolism and response to insulin pathways were found to be significantly affected in all women with ovarian disorders. As shown in FIG. 1, all pathways found to be significantly altered are shown in gray (p-value <0.0125). Additionally, none of the pathways were uniquely altered in the idiopathic group, which suggests that some level of heterogeneity exists in the genetic drivers among patients with idiopathic infertility.

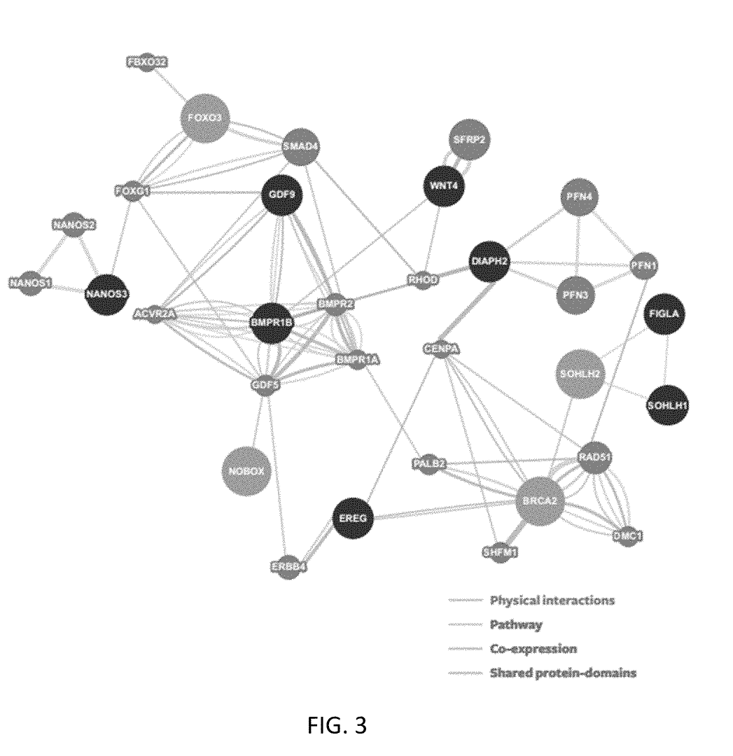

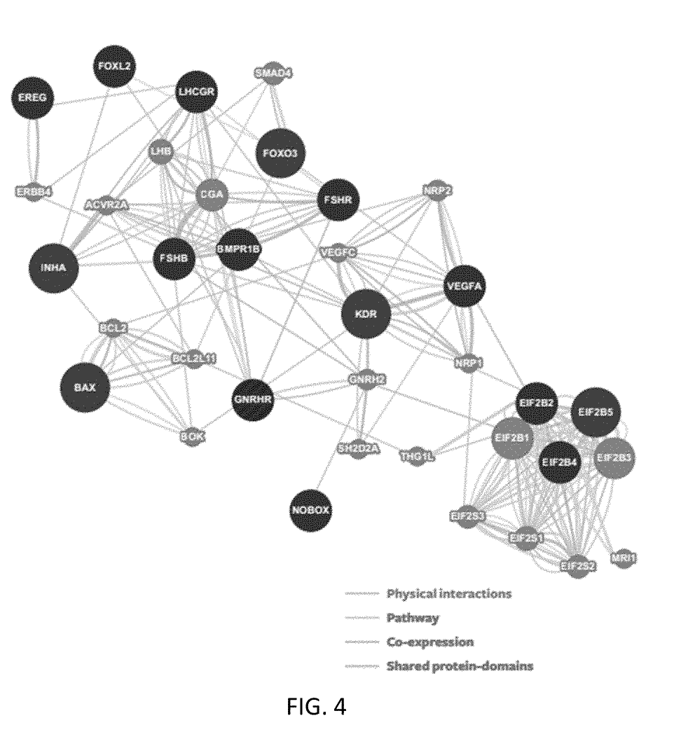

Additionally, specific genes affected in the steroid biosynthesis, oogenesis, and follicular development pathways were investigated. The pathways were enriched with deleterious variants in DOR, PCOS, or both. As shown in FIG. 2, the deleterious variants in the steroidogenesis pathway had a higher frequency in the DOR group compared to the PCOS or control group. For the most part, these variants occurred in key enzymes in the steroidogenic pathway that produce sex hormones, including androgens and estrogens. As shown in FIG. 3, all four genes preferentially altered in PCOS patents were transcription factors directly involved in the regulation of oocyte-specific genes (NOBOX, FOXO3, SOHLH2) or in DNA repair mechanisms (BRCA2). As shown in FIG. 4, the follicle development pathway was altered in PCOS and DOR patients, suggesting that genes within this pathway may be involved in the etiology of both conditions. Table 4 below provides the information contained in FIGS. 2-4 in tabular form. These results are consistent with a previous study by Nilsson et al. showing that gene networks controlling primordial follicle assembly were linked to the etiology of ovarian diseases including POI and PCOS. See Nilsson et al., “Gene bionetworks that regulate ovarian primordial follicle assembly,” BMC Genomics., 14:496 (July 2013).

TABLE 4
Pathways and genes enriched for deleterious variants among different diagnostic groups

| | | | | Variants |
|---|---|---|---|---|
| | Genes involved in | Variants | Variants | with higher |
| | reproductive process | with higher | with higher | frequency in |
| Reproductive | (Variant[s] | frequence in | frequency in | Dor vs control and |
| process | within genes) | DOR vs PCOS | PCOS vs DOR | PCOS vs control |
| Steroid | HSD3B2 (p.R326W) | X | | |
| hormone | CYP1B1 | | | |
| biosynthesis | CYP1A1 | | | |
| | CYP11A1 | | | |
| | HSD17B2 (p.T202M, | X | | |
| | p.G271C, p.R323W) | | | |
| | CYP21A2 (p.A392T) | X | | |
| | CYP11B1 | | | |
| | HSD17B1 | | | |
| | COMT (p.R128H) | X | | |
| | AKR1C3 | X | | |
| | (p.E77G, p.P180S, | | | |
| | p.R258C) | | | |
| | CYP17A1 (p.R21K) | X | | |
| | HSD11B1 | | | |
| | HSD17B3 | | | |
| | SRD5A1 (p.V188I) | X | | |
| | SRD5A2 | | | |
| | CYP19A1 (p.Y241N) | X | | |
| | POR | | | |
| Oogenesis | NANOS3 | | | |
| | WNT4 | | | |
| | EREG | | | |
| | FIGLA | | | |
| | BRCA2 | | X | |
| | (p.N289H, p.N991D, | | | |
| | p.E1593D, p.I3412V) | | | |
| | GDF9 | | | |
| | FOXO3 (p.A341T) | | X | |
| | SOHLH2 (p.G15V) | | X | |
| | SOHLH1 | | | |
| | BMPR1B | | | |
| | NOBOX | | X | |
| | (p.S186Y, p.R163G) | | | |
| | DIAPH2 | | | |
| Follicle | FOXL2 | | | |
| development | LHCGR | | | |
| | FOXO3 (p.G158V) | | | X |
| | INHA (p.S225R) | | | X |
| | FSHR | | | |
| | SOHLH1 | | | |
| | KDR (p.C482R) | | | X |
| | EREG | | | |
| | BAX (p.G39R) | | | X |
| | VEGFA | | | |
| | NOBOX | | | |
| | FSHB | | | |
| | EIF2B2 | | | |
| | BMPR1B | | | |
| | EIF2B4 | | | |
| | EIF2B5 | | | X |
| | (p.D502H, p.R688Q) | | | |
| | GNRHR | | | |

### Discussion

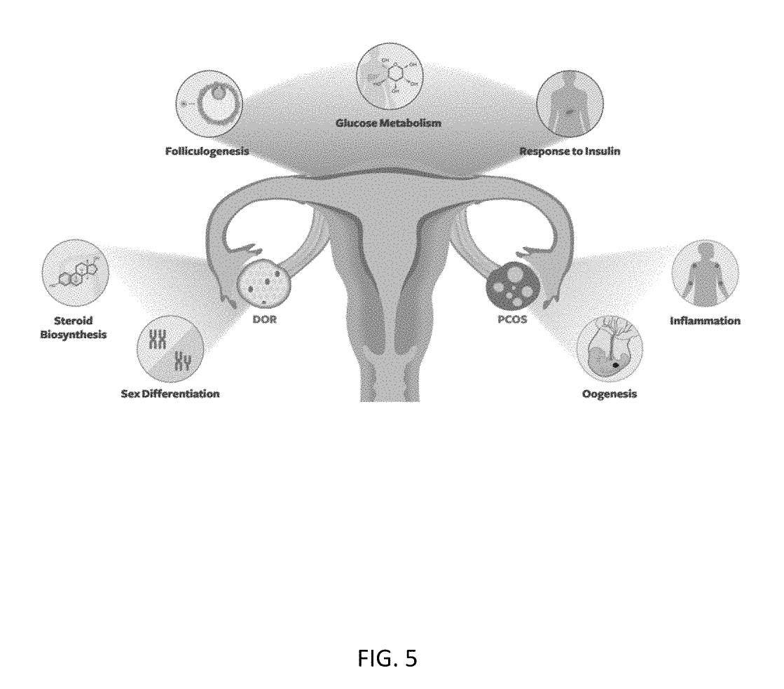

Using whole genome sequencing data, genetic pathways altered in various disorders were identified. As depicted in FIG. 5, the results suggest that clinically distinct ovarian disorders, such as DOR and PCOS, may share common genetic etiologies that affect mechanisms such as follicle development, glucose metabolism, and response to insulin. On the other hand, some pathways are preferentially altered in each condition including steroid biosynthesis and sex differentiation for DOR, and oogenesis and inflammation for PCOS.

Accordingly, these data demonstrate the power of using a pathway level approach, rather than a gene-by-gene, hypothesis driven approach, to identify the genetic drivers of ovarian disorders, particularly in the absence of genetic markers that are unique to each condition. Furthermore, the genetic drivers identified using methods of the invention can be used to help assess the likelihood of abnormal ovarian reserve and/or function, and ultimately fertility, and can also be used to guide course of treatment.

### EXAMPLE 2

Study Design and Methodology 248 patients' genotypes at 47 unique single nucleotide variations (SNVs) were analyzed. Multiple correspondence analysis (MCA), a multivariate dimensionality reduction technique, was first used to identify K <47 principal components which accounted for observed genetic heterogeneity in the sample of 248 patients. Patients were then clustered based on their coordinates in the K-dimensional principal component space. Lastly, differences in clinical metrics between the clusters of patients were tested via t-tests and chi-squared tests where appropriate.

Genotypes at each SNV locus were binary coded according to a dominant genetic model (0=patient did not have the risk allele vs1 =patient was either heterozygous or homozygous for the risk allele) for the initial analysis. The number of principal components retained was determined by utilizing a parallel analysis of the eigenvalues for the dimensions. Briefly, a null distribution for the eigenvalues of the dimensions were generated by performing MCA on several datasets with randomly permuted variants in each patient. The observed eigenvalues were then compared to the 95th percentile of the null distribution and dimensions which were above the 95th percentile were retained.

Upon identifying the number of dimensions, hierarchical clustering of patients based on principal component dimension coordinates was performed. The number of clusters that maximized the relative loss of inertia (within-cluster sum of the squared distance to the cluster centroid) was chosen as the final number of clusters in the patients. Genotypic, phenotypic, clinical, and demographic characteristics were compared between clusters to identify any defining characteristics of cluster membership.

### Results

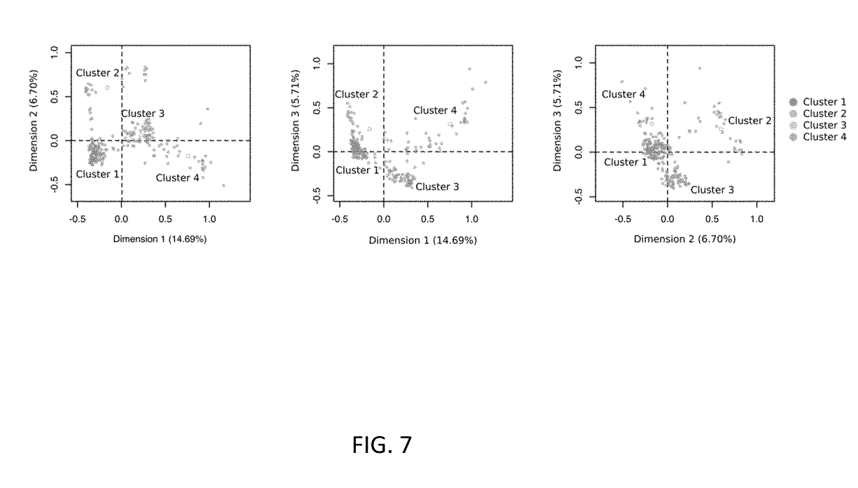

The parallel analysis indicated that the first three principal components from the MCA should be retained. Hierarchical clustering of patients on principal components revealed four distinct clusters of patients in the three dimensional principal component space (FIG. 7). Moreover, several clinical metrics were found to differ between clusters (Tables 5 and 6).

Cluster 1 corresponded to patients who had higher frequencies of risk alleles in the THADA gene, but lower frequencies of risk alleles in the DENND1A gene, relative to the rest of the other clusters. Patients in this cluster were more likely to be classified as low responders, were less likely to have hirsutism or acne, had fewer eggs retrieved, and were more likely to be diagnosed as DOR or POI.

Cluster 2 corresponded to patients who had higher frequencies of risk alleles in the THADA and DENNDIA genes relative to the rest of the clusters. Patients in this cluster were more likely to have a diagnosis of OHSS or PCOS, had more irregular ovulatory cycles, and lower thyroid stimulating hormone (TSH) levels.

Cluster 3 was largely a mixture of patterns observed in Clusters 1 and 2 with no clear pattern of genetic signature. Patients in this cluster were less likely to be classified as low responders relative to the other clusters, had more implantation failures, higher TSH, and lower FSH to LH ratio.

Cluster 4 appeared to correspond to an outlying group and was less likely to have risk alleles in the 47 variants analyzed. Patients in this cluster were less likely to have uterine polyps, and had a higher FSH to LH ratio relative to patients in the other clusters.

TABLE 5
Categorical features which differ between clusters. P-values are from
chi-squared tests. Adjusted p-values are FDR corrected for 118 tests
(all genetic variant tests + all clinical metric tests).

| | | % of | Overall | | |
|---|---|---|---|---|---|
| | | patients | % of | | |
| | | in cluster | patients | | |
| | | with | with | | Adjusted |
| Cluster | Feature | feature | feature | P-value | p-value |
| 1 | Low response | 12.1 | 6.5 | <0.001 | 0.020 |
| | No hirsutism | 39.7 | 29.8 | 0.002 | 0.020 |
| | No acne | 35.3 | 26.2 | 0.002 | 0.020 |
| | DOR or POI | 37.9 | 29.4 | 0.006 | 0.033 |
| | Pelvic pain | 52.6 | 44 | 0.011 | 0.046 |
| 2 | OHSS or PCOS | 7.1 | 0.8 | 0.012 | 0.048 |
| 3 | Low response | 0 | 6.5 | 0.003 | 0.020 |
| 4 | No uterine | | | | |
| | polyps reported | 100 | 88.3 | 0.019 | 0.058 |

TABLE 6
Continuous features which differ between clusters. P-values are from
analysis of variance. Adjusted p-values are FDR corrected for 118 tests
(all genetic variant tests + all clinical metric tests).

| | | Cluster | Overall | | Adjusted |
|---|---|---|---|---|---|
| Cluster | Feature | mean | mean | P-value | p-value |
| 1 | Eggs retrieved | 13.12 | 14.36 | 0.016 | 0.13 |
| 2 | Cycle duration (days) | 37.39 | 32.24 | 0.010 | 0.13 |
| | # of implantation failures | 3.29 | 4.28 | 0.046 | 0.21 |
| | Thyroid stimulating | 1.53 | 2.01 | 0.016 | 0.13 |
| | hormone | | | | |
| 3 | # of failed cycles | 4.62 | 4.01 | 0.020 | 0.14 |
| | (no pregnancy) | | | | |
| | # of of implantation | 4.91 | 4.28 | 0.022 | 0.14 |
| | failures | | | | |
| | Thyroid stimulating | 2.25 | 2.01 | 0.032 | 0.18 |
| | hormone | | | | |
| | FSH to LH ratio | 1.34 | 1.48 | 0.046 | 0.21 |
| 4 | FSH to LH ratio | 1.86 | 1.48 | 0.004 | 0.11 |

Four distinct clusters of patients were identified by a hierarchical clustering algorithm based on the four dimensions uncovered by MCA of the patient's SNVs. One of the clusters corresponded to patients who were more likely to be diagnosed with DOR or POI relative to the rest of the sample, and another cluster corresponded to patients who were more likely to be diagnosed with OHSS or PCOS. These findings provide evidence that patients can be classified as DOR/POI or PCOS based on combinations of SNVs in the variants analyzed.

What is claimed is:

1. A method for analyzing likelihood of ovarian dysfunction, the method comprising of
identifying a plurality of variations in sequence reads from nucleic acid obtained from a female subject;
filtering the plurality of variations into functional biological pathways;
determining the frequency distribution of the variants in each functional biological pathway;
comparing the frequency distributions obtained from each functional biological pathway to an estimated frequency from a reference set; and
determining the likelihood of ovarian dysfunction based upon a comparison of the obtained frequency distributions to the reference set.

2. The method of claim 1, wherein the plurality of variants include SNVs in fertility-centric genes.

3. The method of claim 1, wherein the identifying a plurality of variations comprises:
sequencing nucleic acid from a sample from the female subject to produce sequence reads;
comparing the sequence reads to a reference; and
identifying the plurality of variations in the sequence reads relative to the reference.

4. The method of claim 1, wherein one or more of the functional biological pathways are selected from the group consisting of DNA damage; male sex differentiation; female gonad development; blood circulation; ovulation cycle; oogenesis; glucose metabolism; hormone metabolism; lipid metabolism; response to hormone stimulation; inflammation-autoimmunity; response to wound healing; regulation of cell motion; follicle development; inflammation; immune response; response to insulin; extracellular matrix remodeling; drug metabolism; vasculature development; cell cycle RNA metabolic process; muscle contraction; folic acid; and steroid biosynthesis.

5. A method for analyzing ovarian dysfunction comprising:
identifying a plurality of variations in sequence reads from nucleic acid from a female subject;
clustering subjects based on their patterns of sequence variations; and
identifying phenotypic differences with respect to ovarian dysfunction between these clusters of patients.
